(19) United States
(12) Patent Application Publication
(10) Pub. No.: US 2025/0231076 A1
(43) Pub. Date: Jul. 17, 2025

(54) Gas leak detector

(71) Applicant: QTF Sweden AB, KALMAR (SE)

(72) Inventor: Bjorn CARLSSON, KALMAR (SE)

(21) Appl. No.: 18/856,636

(22) PCT Filed: Apr. 25, 2023
(86) PCT No.: PCT/EP2023/060844

§ 371 Date: Oct. 14, 2024

(30) Foreign Application Priority Data
Apr. 26, 2022 (SE) 2250500-2

(51) Int. Cl. G01M 3/22; G01N 1/22; G01N 33/00; G08B 21/18

(52) U.S. Cl. CPC G01M 3/228; G01N 1/2247; G01N 33/004; G01N 33/0063; G08B 21/182; G01N 2001/2267

(57) ABSTRACT

There is provided a device comprising
a degassing valve,
a conduit for collecting gas released from the degassing valve,
a gas sensor able to detect a gas connected to the conduit and arranged to provide
a signal corresponding to a concentration of the gas,
an alarm device arranged to receive the signal from the gas sensor and to provide
an alarm when the concentration of the gas is above a threshold level.

### FIELD OF THE INVENTION

This invention is related to a device for detecting leaks from gas systems.

### BACKGROUND

Cooling systems typically comprise one subsystem comprising a cooling gas and at least one subsystem containing a liquid, and a heat exchanger arranged to transfer heat between the subsystems. Heating systems may have a similar configuration. Occasionally leaks may occur in the gas subsystem. This will cause gas to escape the system. The system will then have to be repaired and new gas has to be filled in the system. Currently such leaks are not detected until the system fails because of low gas pressure. This may cause large problems. When the cooling system is a part of a freezer in a supermarket, for example, the temperature will rise, the food in the freezer will spoil unless it can be removed and stored elsewhere, and the system has to be urgently repaired. There is a need for a device to detect such leaks earlier and in a more efficient manner.

### SUMMARY OF INVENTION

In a first aspect of the invention there is provided a device comprising a degassing valve, a conduit for collecting gas released from the degassing valve, a gas sensor able to detect a gas connected to the conduit and arranged to provide a signal corresponding to a concentration of the gas, an alarm device arranged to receive the signal from the gas sensor and to provide an alarm when the concentration of the gas is above a threshold level.

By using the device even small amounts of leaked gas can be detected which enables an operator to take action early.

The device may comprise a pressure decreasing device which degasses a liquid at least partly by lowering the pressure. This is useful for detecting gases that dissolve in the liquid, for example carbon dioxide or ammonia.

The gas sensor may preferably a sensor for a gas selected from a hydrocarbon gas, a hydrofluorocarbons gas, a hydrofluoroolefin gas, carbon dioxide, propane, isobutane, ammonia, or mixtures thereof.

In a preferred embodiment the gas sensor is a sensor for carbon dioxide.

In a second aspect of the invention there is provided a system comprising a first subsystem containing a gas, a second subsystem containing a liquid and a heat exchanging interface between the first and second subsystems, the second subsystem having connected thereto a device according to the first aspect of the invention where the gas detector is arranged to detect the gas that is present in the first subsystem.

The gas may be selected from a hydrofluorocarbon (HFC) a hydrofluoroolefin (HFO), carbon dioxide, propane, isobutane, ammonia, or mixtures thereof, in particular carbon dioxide.

The liquid in the second subsystem may be selected from for example water, glycol, brine, alcohol, oil, or aqueous solution of an organic or non-organic salt, or mixtures thereof.

In a third aspect of the invention there is provided a method for detecting gas leakage in a system comprising a first subsystem containing a gas, a second subsystem containing a liquid and a heat exchanging interface between the first and second subsystems, comprising the steps of
a. collecting gas from the liquid in the second subsystem,
b. analyzing the presence of a gas in the gas collected in step a,
c. providing an alarm if the concentration of the gas is above a threshold level.

### DRAWINGS

FIG. 1 shows a system according to the prior art.

FIG. 2 is a schematic drawing of a device.

FIG. 3 is a schematic drawing of system with a device.

FIG. 4 is a schematic drawing of a system with a device.

### DETAILED DESCRIPTION

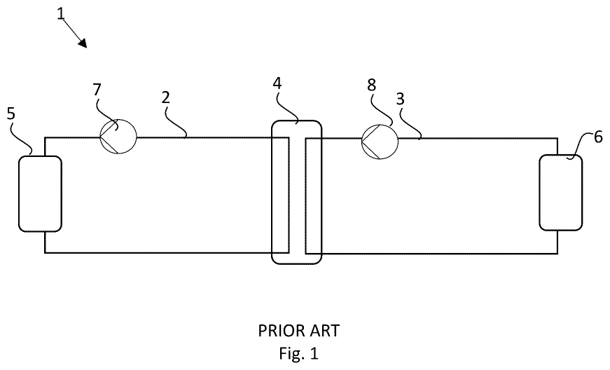

System 1 shown in FIG. 1 is essentially as known in the art. System 1 comprises a gas subsystem 2 (first subsystem) and a liquid subsystem 3 (second subsystem). Typically, gas circulates in the gas subsystem, for example with the use of a first pump 7. Likewise, a liquid circulates in the liquid subsystem 3, typically with the use of a second pump 8. A heat exchanger 4 is arranged to transfer heat between the gas subsystem 2 and the liquid subsystem 3.

The system 1 is typically a heating system or a cooling system. Optional transfer components 5 and 6 may be arranged to cool or heat or to collect or dissipate heat. Often the system 1 may comprise a third subsystem 10 which contains a liquid. Component 5 may be a third subsystem 10 that contains a liquid. In a preferred embodiment system 1 is a cooling system for example for a fridge or a freezer.

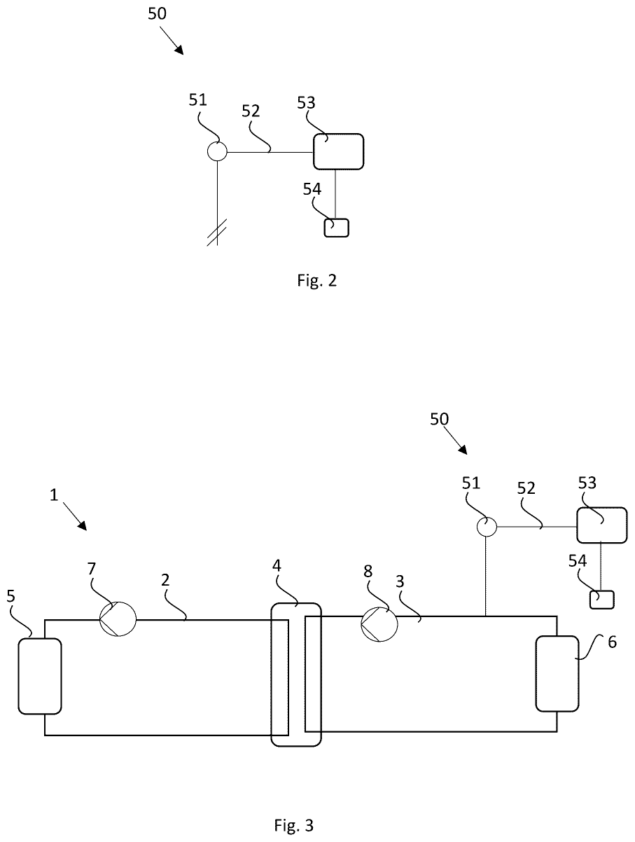

FIG. 2 shows a device 50 according to the present invention. The device 50 comprises a degassing valve 51. Any suitable degassing valve for degassing a liquid may be used. For example, the degassing valve 51 may be comprise a chamber with a body which sinks and opens an outlet when gas is collected in the chamber, and which floats and closes the valve when the gas has been released.

The degassing valve 51 may comprise a device for decreasing the pressure in order to release gas from the liquid in the second system 3. Preferably the pressure is decreased in a continuous manner as the liquid flows. Any suitable type of pressure decreasing device may be used. For example, a venturi type device may be used, or a pump. The pressure decreasing device may be of particular importance when the gas is a gas that dissolves in the liquid or forms a chemical compound with the liquid by reaching with the water at a certain pressure (for example the operating pressure and temperature of the second system 3) but which is a gas at a lower pressure or higher temperature. Examples of such gases include carbon dioxide and ammonia. Carbon dioxide, for example reacts with water to form carbonic acid.

Gas that is released from the degassing valve 51 is led by conduit 52 to a gas sensor 53. At least a part of the gas released from the degassing valve 51 is led to the gas sensor 53. Preferably all the gas is led to the gas sensor 53. After passing gas sensor 53 the gas may be released from the device 50, for example into ambient air.

The gas in the first subsystem 2 is preferably selected from a hydrofluorocarbon (HFC) a hydrofluoroolefin (HFO), carbon dioxide, propane, isobutane, ammonia, or mixtures thereof.

The gas sensor 53 should be arranged to detect at least the gas which is present in the first subsystem 2. The gas sensor 53 may be specific for the gas in the first subsystem 2. For example, the gas sensor may be arranged to detect hydrocarbons when the gas in first subsystem is a HFC or a HFO. Hence the gas sensor may be a gas sensor that detects one chemical compound or a range of chemical compounds, such as a class of structurally related compounds. The gas sensor may hence be specific in the sense that it does not detect other gases to some degree.

In various embodiments the gas sensor 53 may be specific for one gas selected from the following gases: carbon dioxide, ammonia, hydrocarbons, HFC:s or HFO:s. The gas sensor may be arranged to detect the presence in the gas in ambient air.

Any suitable technology for detecting a gas can be used. For example, the gas sensor 53 may comprise an IR light source and an IR sensor arranged to detect the IR light from the light source. A band pass filter may be arranged between the IR light source and the IR sensor. The filter may be chosen depending on the gas to be detected, as every gas has its own specific absorption spectrum. The choice of filter thus can make the gas sensor specific for one gas, as is known in the art. Such gas sensors may be referred to as infrared point sensors. There are suitable filters for CO₂, ammonia, hydrocarbons, etc. Other technologies for detecting specific gases are available, for example such as electrochemical gas detectors.

Ambient air comprises carbon dioxide and may also comprise small amounts of hydrocarbons, and other gases. The threshold should be selected so that an alarm is not provided in normal circumstances. For example, if ambient air is present at the sensor, the threshold should be selected so that ambient levels of the gas does not provide an alarm.

The gas sensor 53 may arranged to provide a signal that is corresponding to a concentration of the specific gas, or indicative that a gas concentration is above a threshold. The signal is provided to an alarm device 54. The alarm device 54 may arranged to provide an alarm signal if the gas concentration is above a predetermined threshold level. When used in system 1, the alarm signal indicates that gas has leaked from the gas subsystem 2 to the liquid subsystem 3. The alarm signal may be provided in any suitable manner. For example, the alarm signal may be provided as a light signal (for example a flashing light) or a sound. The alarm signal may be provided to a network for example to a server or a mobile device. For example, a user may receive an alert in a mobile device. The signal from the sensor 53 may also be provided as data representing a gas concentration. This enables a user to receive detailed information about any gas release from the system 1, in particular leakage from the gas subsystem 2 to the liquid subsystem 3.

In various embodiments, shown in FIG. 3, the device 50 is connected to the liquid subsystem 3 of system 1 such that the presence of gas in liquid subsystem 3 can be detected. A gas leak may often occur in the heat exchanger 4. This will cause gas to enter the liquid subsystem 3 from the gas subsystem 2, in particular when the pressure is higher in the gas subsystem 2 than in the liquid subsystem 3. The heat exchanger 4 may comprise a first space with contains the gas and which is part of first subsystem 2 and a second space which contains the liquid, and which is a part of second subsystem 3 and where the first and second spaces are separated by a wall. The device 50 can be used to detect leakage of gas through the wall.

System 1 may be a system for the transfer of heat, for example a heat pump or other system for extracting heat or cold from for example ambient air, drilled energy wells, bodies of water, waste heat or wastewater. System 1 may be a district heating system, a district cooling system or a large-scale freezer such as freezers in supermarkets. In a preferred embodiment the system 1 is a cooling system for example a system for cooling a fridge or a freezer.

The liquid in the liquid system 3 is typically a liquid used in heat exchange system described herein such as one of water, glycol, brine, alcohol, oil, or an aqueous solution of an organic or non-organic salt, or mixtures thereof.

The gas in the gas subsystem 2 is typically one of the gases mentioned above. If leaks occur in system 1, gas may enter the second subsystem 3 via heat exchanger 4. The device 50 may then provide an alarm to an operator of the system 1. The operator of the system 1 may then take appropriate action such as repairing system 1 in an orderly fashion. Gas leaks may begin as very small leaks that cause the gradual and slow decease in pressure in the gas subsystem 2. But when the gas pressure is below a working range the system 1 will fail. By using device 50 even small amounts of leaked gas can be detected which enables an operator to take action early.

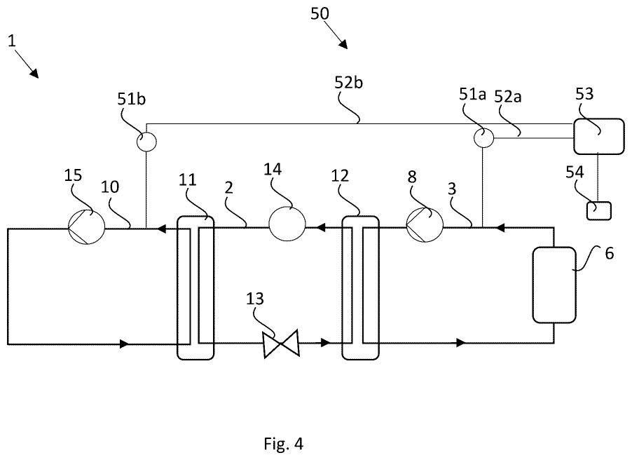

FIG. 4 shows an embodiment with a first subsystem 2 and a second subsystem 3 and a third subsystem 10 which contains a liquid. Here, system 1 may comprise a condenser 11, an evaporator 12 and expansion valve 13 and a compressor 14 and third pump 15. This is an example only and one or more of these components may be omitted. In FIG. 4 the second subsystem is the cold side of the system 1 whereas the third subsystem 10 is the hot side of the system 1.

The system of FIG. 4 may comprise one or more devices 50. In the embodiment shown FIG. 4 each liquid subsystem 3, 10 has a separate degassing valve 51a, 51b and a separate conduit 52a, 52b for leading gas to one single gas detector 53 connected to a single alarm device 54. In other embodiments each of second subsystem 3 and third subsystem 10 has a separate device 50, or only one of second subsystem 3 and third subsystem 10 has a separate device 50.

The alarm device 54 may be arranged in any suitable manner and may use any suitable combination of hardware or software. The alarm device may comprise an A/D converter for converting an analogue signal from the gas sensor 53 to a digital sensor, a bus, a memory and a processor. A threshold may be stored in the memory of the alarm device 54 or in the gas sensor 53. The alarm device 54 may comprise a network interface for providing a digital signal to a second device such as a mobile device.

A method for detecting gas leakage in system 1 comprising a first subsystem 2 containing a gas, a second subsystem 3 containing a liquid and a heat exchanging interface 4 between the first and second subsystems, may comprise the steps of collecting gas from the liquid in the second subsystem, analyzing the presence of a gas in the gas collected in step a, and providing an alarm if the concentration of gas is above a threshold level. The method may comprise the step of decreasing the pressure in the liquid subsystem 3 or a part thereof in order to release gas.

It is realized that everything which has been described in connection to one embodiment is fully applicable to other embodiments, as compatible. Hence, the invention is not limited to the described embodiments, but can be varied within the scope of the enclosed claims. While the invention has been described with reference to specific exemplary embodiments, the description is in general only intended to illustrate the inventive concept and should not be taken as limiting the scope of the invention. The invention is generally defined by the claims.

1. A device being connectable to a heating or cooling system, selected from a heat pump, a district heating system, a district cooling system, a fridge or a freezer, the device comprising:
a degassing valve,
a conduit for collecting gas released from the degassing valve,
a gas sensor able to detect a gas, the gas sensor being connected to the conduit and being arranged to provide a signal corresponding to a concentration of the gas,
an alarm device arranged to receive the signal from the gas sensor and to provide an alarm when the concentration of the gas is above a threshold level, wherein the gas sensor is a sensor specific for a gas selected from; a hydrocarbon gas, a hydro-fluorocarbons gas, a hydrofluoroolefin gas, carbon dioxide, propane, isobutane, ammonia, and mixtures thereof.

2. The device according to claim 1, further comprising a pressure decreasing device which degasses a liquid at least partly by lowering the pressure.

3. The device of claim 1, wherein the sensor is a sensor for carbon dioxide.

4. A system comprising a first subsystem containing a circulating gas, a second subsystem comprising a pump and containing a liquid and a heat exchanging interface between the first and second subsystems, the second subsystem having connected thereto a device comprising
a degassing valve,
a conduit for collecting gas released from the degassing valve,
a gas sensor able to detect a gas connected to the conduit and arranged to provide a signal corresponding to a concentration of the gas,
an alarm device arranged to receive the signal from the gas sensor and to provide an alarm when the concentration of the gas is above a threshold level, wherein the gas detector is arranged to detect the gas that is present in the first subsystem.

5. The system according to claim 4 where the gas in the first subsystem is a gas selected from selected from; a hydrofluorocarbon (HFC) a hydrofluoroolefin (HFO), carbon dioxide, propane, isobutane, ammonia, and mixtures thereof.

6. The system of claim 4, wherein the gas is carbon dioxide.

7. The system according to claim 4, wherein the liquid in the second subsystem is selected from: water, glycol, brine, alcohol, oil, aqueous solution of an organic or non-organic salt, and mixtures thereof.

8. A method for detecting gas leakage in a system comprising a first subsystem containing a circulating gas, a second subsystem comprising a pump and containing a liquid and a heat exchanging inter-face between the first and second subsystems, the method comprising:
a. collecting gas from the liquid in the second subsystem,
b. analyzing the presence of a gas in the gas collected in step a,
c. providing an alarm if the concentration of the gas is above a threshold level.
